(19) United States
(12) Patent Application Publication
(10) Pub. No.: US 2020/0281665 A1
(43) Pub. Date: Sep. 10, 2020

(54) ELECTROMECHANICAL SURGICAL SYSTEMS AND ROBOTIC SURGICAL INSTRUMENTS THEREOF

(71) Applicant: Covidien LP, Mansfield, MA (US)

(72) Inventor: Brock Kopp, Branford, CT (US)

(21) Appl. No.: 16/082,108

(22) PCT Filed: Mar. 3, 2017
(86) PCT No.: PCT/US2017/020563

§ 371 Date: Sep. 4, 2018

Related U.S. Application Data
(60) Provisional application No. 62/303,695, filed on Mar. 4, 2016.

(51) Int. Cl. A61B 34/30; A61B 17/072

(52) U.S. Cl. CPC A61B 34/30; A61B 2017/00017; A61B 17/07207

(57) ABSTRACT

A robotic surgical instrument for actuating an electromechanical end effector includes a housing, a first input drive, a second input drive, and a shaft assembly. The housing has a proximal end configured to be coupled to an instrument drive unit. The first and second input drives are rotatably disposed within the housing and configured to be drivingly coupled to respective first and second motors of the instrument drive unit. The shaft assembly extends distally from within the housing and includes a shaft and a rod. The shaft has a distal end, and a proximal end operably coupled to the first and second input drives. The rod has a proximal end threadingly coupled to the distal end of the shaft. Rotation of the first and second input drives rotates the shaft to effect axial movement of the rod relative to the shaft.

### CROSS-REFERENCE TO RELATED APPLICATIONS

This application claims the benefit of and priority to U.S. Provisional Patent Application No. 62/303,695 filed Mar. 4, 2016, the entire disclosure of which is incorporated by reference herein

### BACKGROUND

Robotic surgical systems have been used in minimally invasive medical procedures. Some robotic surgical systems included a console supporting a surgical robotic arm and a surgical instrument including at least one end effector (e.g., forceps or a grasping tool) mounted to the robotic arm. The robotic arm provided mechanical power to the surgical instrument for its operation and movement. Each robotic arm may have included an instrument drive unit having a plurality motors operatively connected to the surgical instrument.

One motor of the instrument drive unit was used to rotate a threaded rod of the surgical instrument, which in turn, effected the opening and closing of jaws of the end effector and/or the stapling function of the end effector. The speed at which the threaded rod rotated was directly proportional to the rate at which the jaws of the end effector opened and closed. However, existing instrument drive units do not open and close the jaws of the end effector at a desired speed of operation while also providing sufficient torque for performing the stapling and/or cutting functions.

### SUMMARY

In accordance with an aspect of the present disclosure, a robotic surgical instrument for actuating an electromechanical end effector is provided. The robotic surgical instrument includes a housing, a first input drive, a second input drive, and a shaft assembly. The housing has a proximal end configured to be coupled to an instrument drive unit. The first input drive is rotatably disposed within the housing and configured to be drivingly coupled to a first motor of the instrument drive unit. The second input drive is rotatably disposed within the housing and configured to be drivingly coupled to a second motor of the instrument drive unit. The shaft assembly extends distally from within the housing and includes a shaft and a rod. The shaft has a distal end, and a proximal end operably coupled to the first and second input drives. The rod has a proximal end threadingly coupled to the distal end of the shaft. Rotation of the first and second input drives rotates the shaft to effect axial movement of the rod relative to the shaft.

In some embodiments, the shaft of the shaft assembly may define a longitudinal axis, and the first and second input drives may be oriented parallel to and offset from the longitudinal axis.

It is contemplated that each of the first and second input drives may include a gear. The shaft of the shaft assembly may also include a gear, which is in operative engagement with the gear of each of the first and second input drives such that the gear of each of the first and second input drives transfers rotational motion to the gear of the shaft. The gear of the shaft and the gear of each of the first and second input drives may be a spur gear.

It is envisioned that each of the first and second input drives may include a coupler configured to be drivingly coupled to a respective one of the first motor and the second motor of the instrument drive unit.

In some aspects, the proximal end of the rod may be disposed within the distal end of the shaft and may be prevented from rotating as the shaft rotates.

In some embodiments, the robotic surgical instrument may further include an end effector operably coupled to a distal end of the rod of the shaft assembly. The end effector may include a pair of opposing jaw members configured to change a size of a gap therebetween and fire staples therefrom upon axial movement of the rod.

In another aspect of the present disclosure, an electromechanical surgical system for use with a robotic system is provided. The electromechanical surgical system includes an instrument drive unit including a first motor and a second motor, and a robotic surgical instrument. The robotic surgical instrument includes a housing, a first input drive, a second input drive, and a shaft assembly. The housing has a proximal end configured to be coupled to the instrument drive unit. The first input drive is rotatably disposed within the housing and configured to be drivingly coupled to the first motor of the instrument drive unit. The second input drive is rotatably disposed within the housing and configured to be drivingly coupled to the second motor of the instrument drive unit. The shaft assembly extends distally from within the housing. The shaft assembly includes a shaft, and a rod. The shaft has a distal end, and a proximal end operably coupled to the first and second input drives. The rod has a proximal end threadingly coupled to the distal end of the shaft. Rotation of the first and second input drives by actuation of the first and second motors rotates the shaft to effect axial movement of the rod relative to the shaft.

In some embodiments, the shaft of the shaft assembly may define a longitudinal axis, and the first and second input drives of the robotic surgical instrument may be oriented parallel to and offset from the longitudinal axis.

It is contemplated that each of the first and second input drives of the robotic surgical instrument may include a gear. The shaft of the shaft assembly may also include a gear, which is in operative engagement with the gear of each of the first and second input drives such that the gear of each of the first and second input drives transfers rotational motion to the gear of the shaft. The gear of the shaft and the gear of each of the first and second input drives may be a spur gear.

It is envisioned that each of the first and second input drives of the robotic surgical instrument may include a coupler. The instrument drive unit may include a first drive coupler, and a second drive coupler. The first drive coupler may extend from the first motor and be configured to be drivingly coupled to the coupler of the first input drive of the robotic surgical instrument. The second drive coupler may extend from the second motor and be configured to be drivingly coupled to the coupler of the second input drive of the robotic surgical instrument.

In some aspects, the proximal end of the rod may be disposed within the distal end of the shaft and be prevented from rotating as the shaft rotates.

In some embodiments, the robotic surgical instrument may further include an end effector operably coupled to a distal end of the rod of the shaft assembly. The end effector may include a pair of opposing jaw members configured to change a size of a gap therebetween and fire staples therefrom upon axial movement of the rod. The electromechanical surgical system may further include a processor configured to actuate the first motor and the second motor of the instrument drive unit to fire staples from the pair of opposing jaw members. The processor may be configured to independently actuate at least one of the first or second motors of the instrument drive unit to move the pair of opposing jaw members.

In some aspects, the first motor and the second motor may each be configured to produce a maximum torque T such that upon the concurrent actuation of the first motor and the second motor, the first and second motors together produce a maximum torque 2T.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a robotic surgical system including an electromechanical surgical system in accordance with the present disclosure;

FIG. 2 is a perspective view of the electromechanical surgical system of FIG. 1, illustrating a robotic surgical instrument and an instrument drive unit being attached to a surgical robotic arm;

FIG. 3 is a cross sectional view of the instrument drive unit of FIG. 2 illustrating a first motor and a second motor;

FIG. 4 is a perspective view of the robotic surgical instrument of FIG. 2;

FIG. 5 is a cross sectional view, taken along lines 5-5 of FIG. 4, of the robotic surgical instrument; and

FIGS. 6 and 7 are perspective views of a prior art end effector for use with the robotic surgical instrument of the present disclosure.

### DETAILED DESCRIPTION

Embodiments of the presently disclosed robotic surgical system including an electromechanical surgical system for actuating an electromechanical end effector and methods thereof are described in detail with reference to the drawings, in which like reference numerals designate identical or corresponding elements in each of the several views. As used herein the term “distal” refers to that portion of the robotic surgical system, instrument drive unit, robotic surgical instrument, electromechanical end effector, or component thereof, that is further from the user, while the term “proximal” refers to that portion of the robotic surgical system, instrument drive unit, robotic surgical instrument, electromechanical end effector, or component thereof, that is closer to the user.

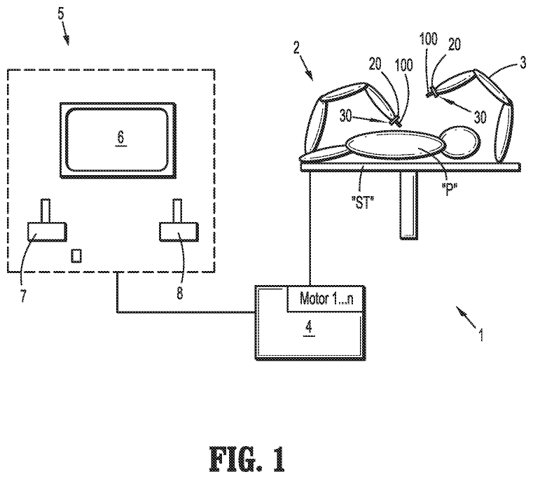

As will be described in detail with respect to FIGS. 1-5, the present disclosure is directed to a surgical instrument, for example, a robotic surgical instrument for use with a robotic surgical system. The robotic surgical instrument includes an instrument drive unit having at least two motors that together drive the actuation of certain functions of an end effector of the robotic surgical instrument, as will be described in detail below. In some embodiments, a handheld surgical instrument, such as, for example, a handheld surgical stapling apparatus, may be provided that has a plurality of motors that together drive a single firing rod of the handheld surgical stapling apparatus.

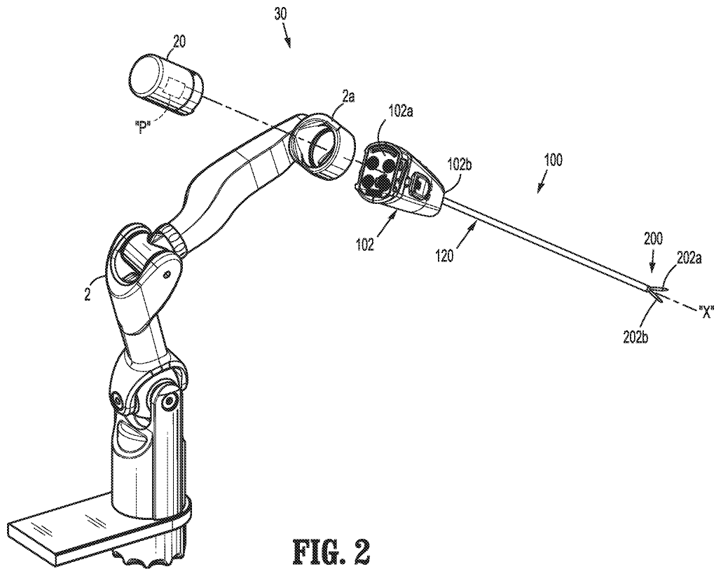

Referring initially to FIGS. 1 and 2, a surgical system, such as, for example, a robotic surgical system 1, generally includes a plurality of surgical robotic arms 2, 3 having a robotic surgical instrument 100 removably attached thereto; a control device 4; and an operating console 5 coupled with control device 4.

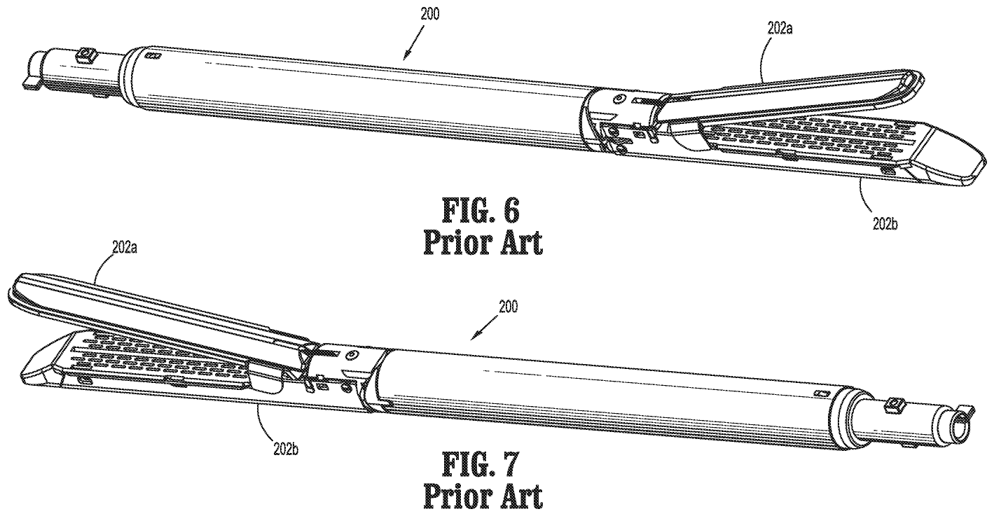

Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art. Each of the robotic arms 2, 3 may be composed of a plurality of members, which are connected through joints. Robotic arms 2, 3 may be driven by electric drives (not shown) that are connected to control device 4. Control device 4 (e.g., a computer) is set up to activate the drives, in particular by means of a computer program, in such a way that robotic arms 2, 3, their instrument drive units 20, and thus robotic surgical instrument 100 (including electromechanical end effector 200, FIGS. 6 and 7) execute a desired movement according to a movement defined by means of manual input devices 7, 8. Control device 4 may also be set up in such a way that it regulates the movement of robotic arms 2, 3 and/or of the drives.

Robotic surgical system 1 is configured for use on a patient “P” lying on a surgical table “ST” to be treated in a minimally invasive manner by means of a surgical instrument, e.g., robotic surgical instrument 100. Robotic surgical system 1 may also include more than two robotic arms 2, 3, the additional robotic arms likewise being connected to control device 4 and being telemanipulatable by means of operating console 5. A surgical instrument, for example, robotic surgical instrument 100 (including electromechanical end effector 200, FIGS. 6 and 7), may also be attached to the additional robotic arm.

Control device 4 may control a plurality of motors (Motor 1 . . . n) with each motor configured to drive a relative rotation of drive members of robotic surgical instrument 100 to effect operation and/or movement of each electromechanical end effector 200 of robotic surgical instrument 100. It is contemplated that control device 4 coordinates the activation of the various motors (Motor 1 . . . n) to coordinate a clockwise or counter-clockwise rotation of drive members (not shown) of instrument drive unit 20 in order to coordinate an operation and/or movement of a respective electromechanical end effector 200. In embodiments, each motor can be configured to actuate a drive rod or a lever arm to effect operation and/or movement of each electromechanical end effector 200 of robotic surgical instrument 100.

For a detailed discussion of the construction and operation of a robotic surgical system, reference may be made to U.S. Pat. No. 8,828,023, entitled “Medical Workstation,” the entire contents of which are incorporated herein by reference.

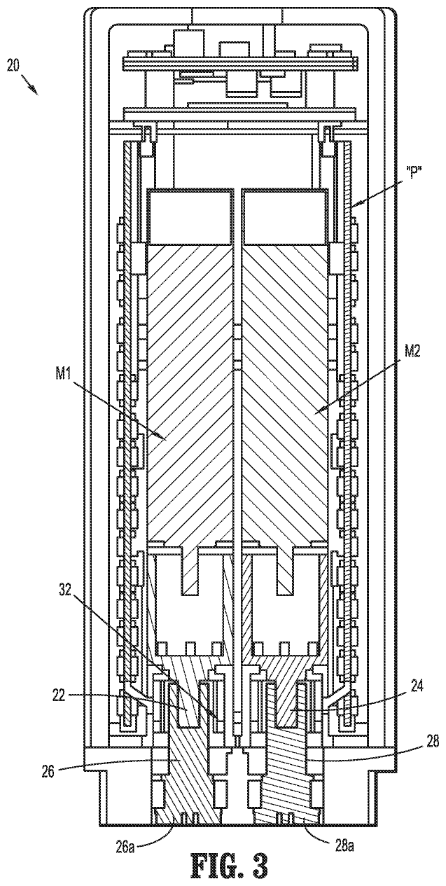

With reference to FIGS. 2 and 3, robotic surgical system 1 includes an electromechanical surgical system 30, which includes robotic arm 2, instrument drive unit 20, and robotic surgical instrument 100. Instrument drive unit 20 of electromechanical surgical system 30 is configured to be coupled to robotic surgical instrument 100, and robotic surgical instrument 100 is configured to be coupled with or to robotic arm 2. Instrument drive unit 20 is configured for powering robotic surgical instrument 100. Instrument drive unit 20 transfers power and actuation forces from its motors, for example, a first motor M1 and a second motor M2, to robotic surgical instrument 100 to ultimately drive movement of components of electromechanical end effector 200 (FIGS. 6 and 7) of robotic surgical instrument 100, for example, a movement of a knife blade (not shown) and/or a closing and opening of jaw members 202a, 202b of electromechanical end effector 200.

First motor M1 may be configured as a master motor, and second motor M2 may be configured as a slave motor that matches the amount of torque being output by master motor M1 so that first and second motors M1, M2 operate in synchrony. First and second motors M1, M2 are in communication with one another via a processor “P” that synchronizes first and second motors M1, M2 so that second motor M2 will produce the same torque as first motor M1 at any given time to ultimately rotate first and second input drives 108, 110 of robotic surgical instrument 100 at the same rate. First and second motors M1, M2 are each configured to produce a maximum torque T, depending on their size and make, such that upon the concurrent actuation of first and second motors M1, M2, first and second motors M1, M2 together produce a maximum torque 2T. In some embodiments, instrument drive unit 20 may include a plurality of slave motors such that instrument drive unit 20 can produce a torque greater than 2T.

In embodiments, when a particular amount of torque is desired to be output by the instrument drive unit 20 (e.g., as determined by a clinician or the control device 4), the processor may be configured to cause the second motor M2 to output a torque that is equal to the difference between the desired torque and the torque output by the first motor M1 such that the combined torque output by the first and second motors M1, M2 matches the desired torque. In embodiments, the second motor M2 may be configured to output a constant torque whereas the first motor M1 may be configured to output an amount of torque that brings the total torque output by the instrument drive unit 20 up to the desired torque.

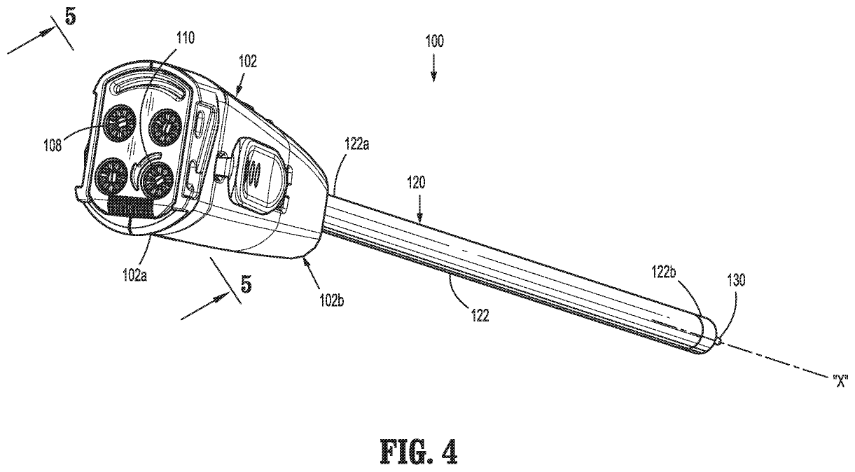

Instrument drive unit 20 includes a plurality of rotatable output shafts 22, 24 attached to respective first and second motors M1, M2 such that output shafts 22, 24, are independently rotatable with respect to one another. In some embodiments, instrument drive unit 20 may include more than two motors, for example, three or four motors, that each have a respective output shaft rotatably attached thereto. In embodiments, the first motor M1 may be the master motor and two or more motors may act as slave motors. Instrument drive unit 20 has a first drive coupler 26 and a second drive coupler 28 non-rotatably attached to respective first and second output shafts 22, 24 such that first and second drive couplers 26, 28 extend from first and second motors M1, M2, respectively. First and second drive couplers 26, 28 each have a mechanical interface 26a, 28a, for example, a plurality of teeth or a crown gear, configured to drivingly couple to respective first and second input drives 108, 110 (FIG. 4) of robotic surgical instrument 100. As such, actuation of first and second motors M1, M2 effects rotation of first and second input drives 108, 110 of robotic surgical instrument 100 at the same rate as one another when robotic surgical instrument 100 is operably engaged to instrument drive unit 20, as will be described in detail below.

Instrument drive unit 20 includes sensors, such as, for example, torque transducers 32, connected to first and second motors M1, M2. Torque transducers 32 sense the amount of torque that is being output by motors M1, M2 during their operation. Processor “P” of instrument drive unit 20 is in communication with torque transducers 32 to control the amount of power output by first and/or second motors M1, M2 based on the amount of torque sensed by torque transducers 32. In particular, when additional torque is required to carry out a certain function of end effector 200, for example, stapling tissue and/or cutting tissue, processor “P” will activate second motor M2 (to operate concurrently with first motor M1) and cause second motor M2 to produce the same torque as first motor M1.

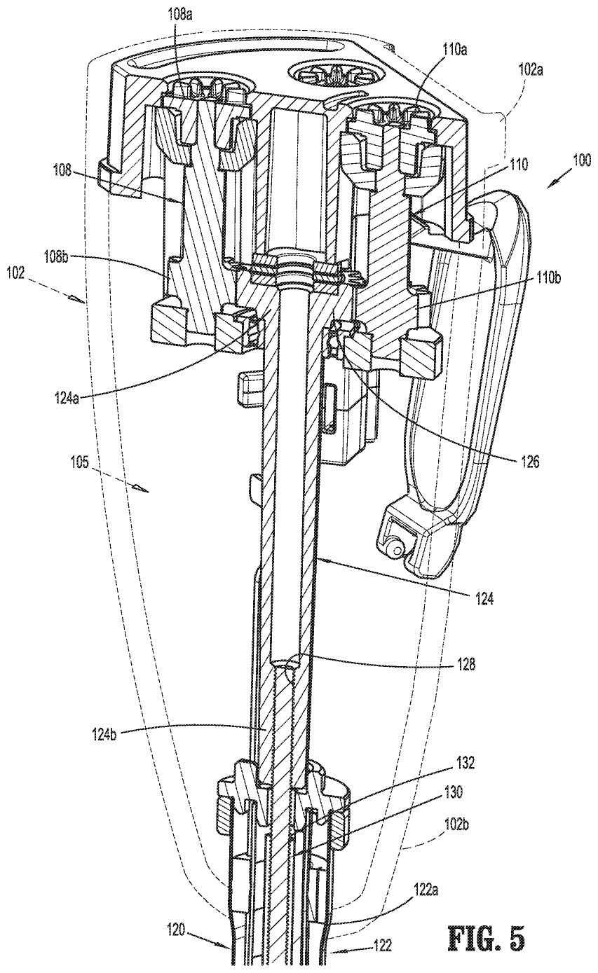

Further, instrument drive unit 20 includes a sensor (e.g. a pressure sensor) (not shown) able to detect and measure both firing and retraction forces of shaft assembly 120 (FIG. 5) of robotic surgical instrument 100. Processor “P” is in communication with the pressure sensor and is configured to actuate both first and second motors M1, M2 concurrently when the amount of force sensed by the pressure sensor is indicative of tissue being clamped and ready for stapling. Processor “P” is also configured to actuate only one first motor M1 when the amount of force sensed by the pressure sensor is indicative of tissue not being clamped between jaws 202a, 202b of electromechanical end effector 200.

As such, a torque T is output by instrument drive unit 20 for clamping and unclamping tissue disposed between jaws 202a, 202b of electromechanical end effector 200, and a torque 2T is output by instrument drive unit 20 for stapling and/or cutting tissue clamped between jaws 202a, 202b of electromechanical end effector 200. It is contemplated that torque transducers 32, the pressure sensors, and/or processor “P” may be disposed in any of the components of electromechanical surgical system 30. It is contemplated that a clinician may activate first motor M1, second motor M1, or first and second motors M1, M2 concurrently depending on the desired effect on electromechanical end effector 200, for example, clamping/unclamping or stapling/cutting. In some embodiments, the instrument drive unit 20 may be configured to output more or less than the torque 2T for stapling and/or cutting tissue.

With reference to FIGS. 4 and 5, robotic surgical instrument 10 generally includes robotic surgical instrument 100, and electromechanical end effector 200, which extends distally from robotic surgical instrument 100.

Robotic surgical instrument 100 includes a housing 102 and a shaft assembly 120 extending distally from within housing 102. Housing 102 of robotic surgical instrument 100 has a generally cylindrical configuration, and has a proximal end 102a configured to be coupled to instrument drive unit 20, and a distal end 102b. In embodiments, housing 102 may be any shape suitable for receipt in a distal end 2a of robotic arm 2. Housing 102 defines a cavity 105 that houses various components of robotic surgical instrument 100. Proximal end 102a of housing 102 supports a first input drive 108 and a second input drive 110 each being rotatably disposed within cavity 105 of housing 102 and extending in parallel alignment with a longitudinal axis “X” defined by shaft assembly 120. In some embodiments, housing 102 may include more than two input drives. First and second input drives 108, 110 of robotic surgical instrument 100 are illustrated as being rod-shaped, but it is contemplated that they may take on any other suitable shape.

First and second input drives 108, 110 of robotic surgical instrument 100 each have a proximal end and a distal end. The proximal end of each of first and second input drives 108, 110 includes a proximal coupler 108a, 110a, for example, a crown gear, disposed at proximal end of housing 102a. Proximal coupler 108a, 110a of each of first and second input drives 108, 110 is configured to be detachably, non-rotatably coupled to mechanical interface 26a, 28a (FIG. 3) of respective first and second drive couplers 26, 28 of instrument drive unit 20. As such, upon connecting instrument drive unit 20 with housing 102 of robotic surgical instrument 100, first and second input drives 108, 110 of robotic surgical instrument 100 are drivingly coupled to respective first and second motors M1, M2 of instrument drive unit 20. In some embodiments, proximal couplers 108a, 110a of robotic surgical instrument 100 may be connected to respective first and second drive couplers 26, 28 of instrument drive unit 20 via helical gears, a belt drive assembly, or any other suitable mechanism for transferring rotational motion between first and second input drives 108, 110 and instrument drive unit 20. The distal end of each of the first and second input drives 108, 110 includes a distal coupler 108b, 110b, for example, a spur gear. Distal coupler 108b, 110b of each of first and second input drives 108, 110 of robotic surgical instrument 100 is in meshing engagement with a gear 126 of shaft assembly 120 of robotic surgical instrument 100.

Thus, upon the concurrent actuation of first and second motors M1, M2 of instrument drive unit 20, first and second drive couplers 26, 28 of instrument drive unit 20 rotate, resulting in concomitant rotation of first and second input drives 108, 110 of robotic surgical instrument 100 via the first and second proximal couplers 108a, 110a of housing 102. The rotation of first input drive 108 and/or second input drive 110 of housing 102 of robotic surgical instrument 100 drives a rotation of an inner shaft 124 of shaft assembly 120 to ultimately result in the opening or closing of jaw members 202a, 202b of electromechanical end effector 200, the ejection of staples (not shown) from jaw members 202a, 202b, and/or the actuation of a knife blade (not shown) of electromechanical instrument 200. In some embodiments, distal couplers 108b, 110b of robotic surgical instrument 100 may be connected to shaft assembly 120 via helical gears, a belt drive assembly, or any other suitable mechanism for transferring rotational motion between first and second input drives 108, 110 and shaft assembly 120.

In some embodiments, second input drive 110 is movable between a first position, in which distal coupler 110b of second input drive 110 is out of meshing engagement with gear 126 of inner shaft 124, and a second position, in which distal coupler 110b of second input drive 110 is in meshing engagement with gear 126 of inner shaft 124. As such, when more torque is required to actuate functions of electromechanical end effector 200, second input drive 110 may be moved from the first position into the second position. When the added torque is not required, second input drive 110 may be moved into the first position.

As mentioned above, robotic surgical instrument 100 includes shaft assembly 120, which extends distally from within housing 102. Shaft assembly 120 operatively intercouples instrument drive unit 20 with jaw members 202a, 202b of electromechanical end effector 200 and a staple actuator (not shown) of electromechanical end effector 200. Shaft assembly 120 generally includes an outer tube or outer shaft 122, an inner shaft 124, and a threaded rod 130. Outer shaft 122 has a proximal end 122a, and a distal end 122b, which is mechanically attached to one or both jaw members 202a, 202b of electromechanical end effector 200.

Inner shaft 124 of shaft assembly 120 has a proximal end 124a and a distal end 124b. Proximal end 124a of inner shaft 124 has a gear 126, for example, a spur gear, in meshing engagement with both distal couplers 108b, 110b of respective first and second input drives 108, 110 of housing 102 such that distal couplers 108b, 110b of first and second input drives 108, 110 transfer rotational motion to gear 126 of inner shaft 124. Distal end 124b of inner shaft 124 defines a threaded bore 128 longitudinally therethrough. Rod 130 of shaft assembly 120 has a threaded outer surface 132 threadingly engaged to threaded bore 128 of inner shaft 124. Rod 130 of shaft assembly 120 has a non-circular portion (not shown) that is disposed within a correspondingly shaped fixture (not explicitly shown) that prevents rod 130 from rotating. As such, as shaft 124 of shaft assembly 120 rotates, rod 130 of shaft assembly 120 does not rotate therewith, but instead, translates or moves axially relative to shaft 124.

Threaded outer surface 132 of rod 130 has a high thread pitch of approximately 32 threads per inch of length of rod 130. The high thread pitch of threaded outer surface 132 of rod 130 provides for a high rate of axial movement of rod 130 per revolution of shaft 124, which ultimately results in a high rate of opening and closing of jaw members 202a, 202b of electromechanical end effector 200.

Rod 130 extends from distal end 102b of housing 102, through the length of outer shaft 122, and terminates at jaw members 202a, 202b of electromechanical end effector 200. The distal end (not shown) of rod 130 is operably coupled to components of end effector 200 such that axial movement of rod 130 effects an opening or closing of jaw members 202a, 202b of electromechanical end effector 200 and the operation of the stapling function and cutting function of electromechanical end effector 200.

For a detailed discussion of the construction and operation of end effector 200, reference may be made to U.S. Pat. No. 6,953,139, filed on Nov. 5, 2004, entitled “SURGICAL STAPLING APPARATUS,” the entire content of which is incorporated herein by reference.

In use, to change a size of a gap between jaw members 202a, 202b of electromechanical end effector 200, instrument drive unit 20 is operably coupled to robotic surgical instrument 100. First motor M1 of instrument drive unit 20 is then activated to drive a rotation of first output shaft 22 of instrument drive unit 20. Rotation of first output shaft 22 effects rotation of first input drive 108 of robotic surgical instrument 100 via the meshing engagement between mechanical interface 26a of first drive coupler 26 of instrument drive unit 20 and proximal coupler 108a of first input drive 108 of robotic surgical instrument 100. Rotation of first input drive 108 of robotic surgical instrument 100 drives either a clockwise or counter-clockwise rotation of inner shaft 124 of shaft assembly 120 via the meshing engagement of distal coupler 108b of first input drive 108 and gear 126 of inner shaft 124.

The rotation of inner shaft 124 causes rod 130 of shaft assembly 120 to move axially relative to shaft 124 in a proximal or distal direction. Proximal axial movement of rod 130 relative to shaft 124 actuates a closing of jaw members 202a, 202b of electromechanical end effector 200, and distal axial movement of rod 130 relative to shaft 124 actuates an opening of jaw members 202a, 202b of electromechanical end effector 200. In some embodiments, distal axial movement of rod 130 may close jaw members 202a, 202b, and proximal axial movement of rod 130 may open jaw members 202a, 202b. As mentioned above, due to the high thread pitch of rod 130, jaw members 202a, 202b open and close at a fast rate.

With tissue clamped between jaw members 202a, 202b, staples may be ejected from electromechanical end effector 200 into the tissue and the knife blade of electromechanical end effector 200 may be translated through the tissue to carry out a particular surgical procedure. There is an increased resistance to rotation of inner shaft 124 of shaft assembly 120 from the tissue clamped between jaws 202a, 202b, and the increased thread pitch of rod 130. Thus, to carry out the stapling function and/or cutting function of electromechanical end effector 200, more torque than what first motor M1 alone can provide may be required.

To staple tissue clamped between jaw members 202a, 202b, a sufficient amount of power is delivered to second motor M2 of instrument drive unit 20 to cause second motor M2 to match the torque output by first motor M1 so that second input drive 110 rotates at the same rate as first input drive 108 and no slip occurs between distal coupler 110b of second input drive 110 and gear 126 of inner shaft 124. Rotation of second input drive 110 of robotic surgical instrument 100 supplements the torque applied to inner shaft 124 of shaft assembly 120 by first input drive 108. Since the rotation of inner shaft 124 of shaft assembly 120 is being driven by both first and second input drives 108, 110, which is being driven by the activation of first and second motors M1, M2, any resistance experienced by electromechanical end effector 200 to stapling through the tissue or to movement of the knife blade through the tissue can be overcome by the added torque provided by second motor M2. It is contemplated that both first and second motors M1, M2 may be activated to open and close jaw members 202a, 202b instead of only first motor M1.

In some embodiments, the shaft assembly may be incorporated into a surgical instrument that uses a capstan/wire spool mechanism for converting rotary motion into linear motion. For example, in this embodiment, the gear 126 of the inner shaft 124 may be configured as a capstan having a wire(s) or cable(s) wrapped thereabout.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A robotic surgical instrument for actuating an electromechanical end effector, the robotic surgical instrument comprising:
a housing having a proximal end configured to be coupled to an instrument drive unit;
a first input drive rotatably disposed within the housing and configured to be drivingly coupled to a first motor of the instrument drive unit;
a second input drive rotatably disposed within the housing and configured to be drivingly coupled to a second motor of the instrument drive unit; and
a shaft assembly extending distally from within the housing, the shaft assembly including:
a shaft having a distal end, and a proximal end operably coupled to the first and second input drives; and
a rod having a proximal end threadingly coupled to the distal end of the shaft, wherein rotation of the first and second input drives rotates the shaft to effect axial movement of the rod relative to the shaft.

2. The robotic surgical instrument according to claim 1, wherein the shaft of the shaft assembly defines a longitudinal axis, and wherein the first and second input drives are oriented parallel to and offset from the longitudinal axis.

3. The robotic surgical instrument according to claim 1, wherein each of the first and second input drives includes a gear, and wherein the shaft of the shaft assembly includes a gear in operative engagement with the gear of each of the first and second input drives such that the gear of each of the first and second input drives transfers rotational motion to the gear of the shaft.

4. The robotic surgical instrument according to claim 3, wherein the gear of the shaft and the gear of each of the first and second input drives is a spur gear.

5. The robotic surgical instrument according to claim 3, wherein each of the first and second input drives includes a coupler configured to be drivingly coupled to a respective one of the first motor and the second motor of the instrument drive unit.

6. The robotic surgical instrument according to claim 1, wherein the proximal end of the rod is disposed within the distal end of the shaft and is prevented from rotating as the shaft rotates.

7. The robotic surgical instrument according to claim 1, further comprising an end effector operably coupled to a distal end of the rod of the shaft assembly, the end effector including a pair of opposing jaw members configured to change a size of a gap therebetween and fire staples therefrom upon axial movement of the rod.

8. The robotic surgical instrument according to claim 7, wherein each of the first and second input drives includes a gear, and wherein the shaft of the shaft assembly includes a gear in operative engagement with the gear of each of the first and second input drives such that the gear of each of the first and second input drives transfers rotational motion to the gear of the shaft.

9. An electromechanical surgical system for use with a robotic system, the electromechanical surgical system comprising:
an instrument drive unit including a first motor and a second motor; and
a robotic surgical instrument including:
a housing having a proximal end configured to be coupled to the instrument drive unit;
a first input drive rotatably disposed within the housing and configured to be drivingly coupled to the first motor of the instrument drive unit;
a second input drive rotatably disposed within the housing and configured to be drivingly coupled to the second motor of the instrument drive unit; and
a shaft assembly extending distally from within the housing, the shaft assembly including:
a shaft having a distal end, and a proximal end operably coupled to the first and second input drives; and
a rod having a proximal end threadingly coupled to the distal end of the shaft, wherein rotation of the first and second input drives by actuation of the first and second motors rotates the shaft to effect axial movement of the rod relative to the shaft.

10. The electromechanical surgical system according to claim 9, wherein the shaft of the shaft assembly defines a longitudinal axis, and wherein the first and second input drives of the robotic surgical instrument are oriented parallel to and offset from the longitudinal axis.

11. The electromechanical surgical system according to claim 9, wherein each of the first and second input drives of the robotic surgical instrument includes a gear, and wherein the shaft of the shaft assembly includes a gear in operative engagement with the gear of each of the first and second input drives such that the gear of each of the first and second input drives transfers rotational motion to the gear of the shaft.

12. The electromechanical surgical system according to claim 11, wherein the gear of the shaft and the gear of each of the first and second input drives is a spur gear.

13. The electromechanical surgical system according to claim 9, wherein each of the first and second input drives of the robotic surgical instrument includes a coupler, and wherein the instrument drive unit includes:
a first drive coupler extending from the first motor and configured to be drivingly coupled to the coupler of the first input drive of the robotic surgical instrument; and
a second drive coupler extending from the second motor and configured to be drivingly coupled to the coupler of the second input drive of the robotic surgical instrument.

14. The electromechanical surgical system according to claim 9, wherein the proximal end of the rod is disposed within the distal end of the shaft and is prevented from rotating as the shaft rotates.

15. The electromechanical surgical system according to claim 9, wherein the robotic surgical instrument further includes an end effector operably coupled to a distal end of the rod of the shaft assembly, the end effector including a pair of opposing jaw members configured to change a size of a gap therebetween and fire staples therefrom upon axial movement of the rod.

16. The electromechanical surgical system according to claim 15, further comprising a processor configured to actuate the first motor and the second motor of the instrument drive unit to fire staples from the pair of opposing jaw members.

17. The electromechanical surgical system according to claim 16, wherein the processor is configured to independently actuate at least one of the first or second motors of the instrument drive unit to move the pair of opposing jaw members.

18. The electromechanical surgical system according to claim 9, wherein the first motor and the second motor are each configured to produce a maximum torque T such that upon concurrent actuation of the first motor and the second motor, the first and second motors together produce a maximum torque 2T.

19. The electromechanical surgical system according to claim 9, wherein the first motor is configured as a master motor, and the second motor is configured as a slave motor that matches the amount of torque being output by the master motor such that that the master and slave motors operate in synchrony.
